(19) United States
(12) Patent Application Publication
(10) Pub. No.: US 2017/0239435 A1
(43) Pub. Date: Aug. 24, 2017

(54) TRACHEAL TUBES AND CUFFS

(71) Applicant: SMITHS MEDICAL INTERNATIONAL LIMITED, Kent (GB)

(72) Inventors: Timothy Bateman, Kent (GB); Stephen James Field, Kent (GB); Mark Andrew Graham, Kent (GB); Andrew Thomas Jeffrey, Kent (GB)

(73) Assignee: SMITHS MEDICAL INTERNATIONAL LIMITED, Kent (GB)

(21) Appl. No.: 15/521,634

(22) PCT Filed: Sep. 25, 2015
(86) PCT No.: PCT/GB2015/000272

§ 371 Date: Apr. 25, 2017

(30) Foreign Application Priority Data
Oct. 28, 2014 (GB) 1419141.5

(51) Int. Cl. A61M 16/04

(52) U.S. Cl. CPC A61M 16/0445; A61M 16/0465; A61M 16/0488

(57) ABSTRACT

A tracheal tube has a high-volume/low-pressure sealing cuff (10, 201, 310, 510, 610, 710, 810) with an outer surface provided with surface formations (211, 311, 511, 611, 711, 811) adapted to reduce the flow of secretions past the cuff. The formations could be in the form of an array of dimples (211) or a pattern of linear ridges or channels (611 or 711). Alternatively, the surface formations, could be a pattern of intersecting linear projections (311, 312, 313) or grooves (511). Instead the surface formations could be provided by a rough texture (811) on the outer wall surface.

This invention relates to tracheal tubes of the kind having a shaft and an inflatable sealing cuff mounted on the shaft towards its patient end.

Tracheal tubes are used to supply ventilation and anaesthetic gases to a patient, such as during surgery. The tracheal tube may be inserted via the mouth or nose, in the case of an endotracheal tube, or may be inserted via a surgically-made tracheostomy opening in the neck, in the case of a tracheostomy tube. Most, but not all, tracheal tubes have some form of a seal on their outside which forms a seal between the outside of the tube and the inside of the trachea so that gas flow is confined to the bore of the tube and cannot flow around the outside of the tube, between the tube and the trachea.

The most common form of seal is provided by an inflatable cuff that is inflated and deflated via a small bore lumen extending along the tube and connected towards its rear end to an inflation line terminated by an inflation indicator, valve and connector. These inflatable cuffs may be of the high-volume/low-pressure kind where the cuff is formed of a flexible plastics material moulded with a natural annular or doughnut shape that is inflated without stretching, to contact the wall of the trachea, by relatively low-pressure gas supplied via the inflation line. Alternatively, the cuff may be of the low-volume/high-pressure kind where the cuff is of an elastic material that lies close to the tube shaft when uninflated but is inflated and stretched to a larger diameter by relatively high pressure gas supplied via the inflation line. Various problems exist with both forms of cuff. One problem is the difficulty of preventing secretions that collect above the cuff leaking between the cuff and the trachea and entering the bronchial passages. The leakage of such secretions is thought to contribute to ventilator-associated pneumonia (VAP).

Various suggestions have been made for cuffs intended to reduce leakage. U.S. Pat. No. 6,526,977 and U.S. Pat. No. 6,802,317 describe thin sealing cuffs of polyurethane.

It is an object of the present invention to provide an alternative tracheal tube and cuff.

According to one aspect of the present invention there is provided a tracheal tube of the above-specified kind, characterised in that the cuff has a wall with an outer surface provided with surface formations adapted to reduce the flow of secretions past the cuff.

The surface formations may be provided by an array of dimples or a pattern of linear ridges or channels on the wall surface. In other embodiments the surface formations could be provided by a pattern of intersecting linear projections or grooves on the wall surface. The dimensions of the surface formations may be between 0.1 mm and 1 mm. In a further alternative embodiment the surface formations could be provided by a rough texture on the outer wall surface. The cuff is preferably of the high-volume/low-pressure kind.

According to another aspect of the present invention there is provided a cuff for a tube according to the above one aspect of the present invention.

A tracheostomy tube according to the present invention will now be described, by way of example, with reference to the accompanying drawings, in which:

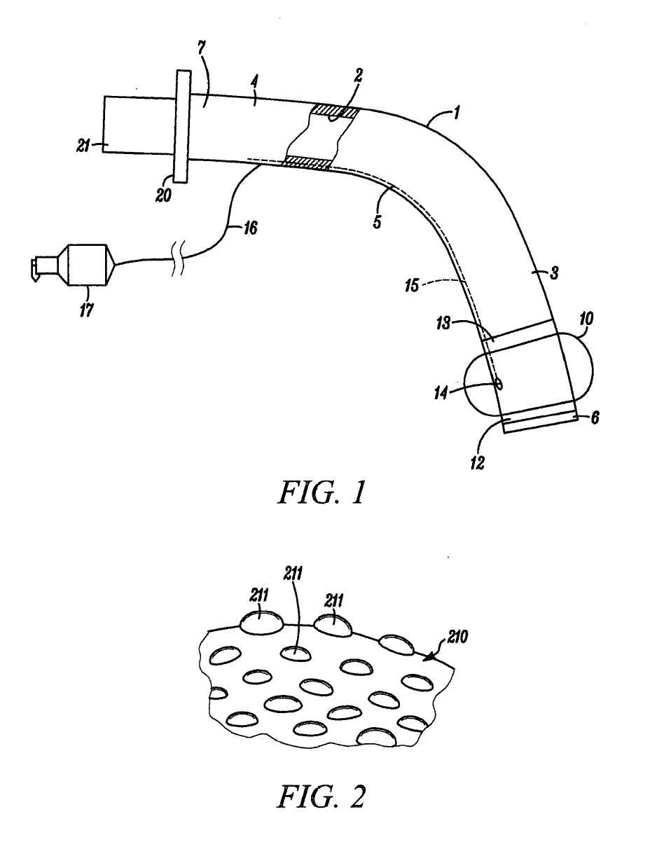

FIG. 1 is a side elevation view of the tube;

FIG. 2 is an enlarged perspective view showing a part of a first form of the cuff of the tube;

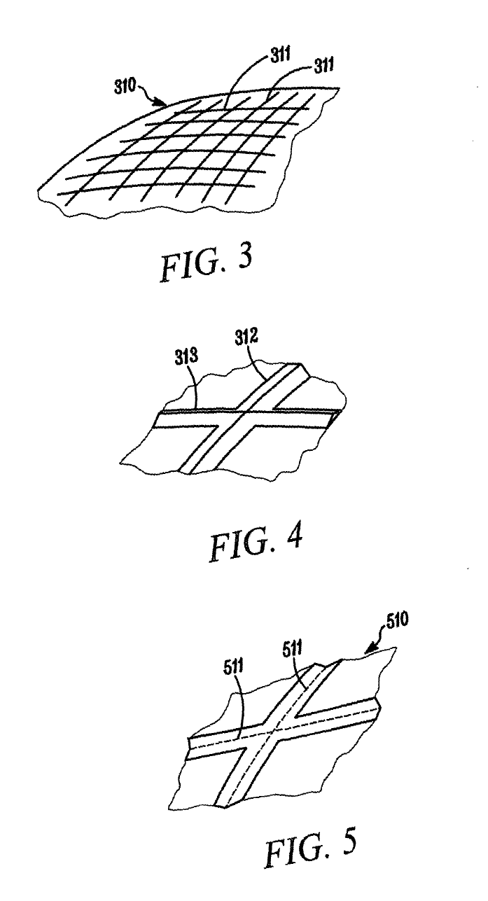

FIG. 3 is an enlarged perspective view showing a part of a second form of cuff;

FIG. 4 is a further enlargement of the cuff shown in FIG. 3;

FIG. 5 is an enlargement of a part of a third form of cuff;

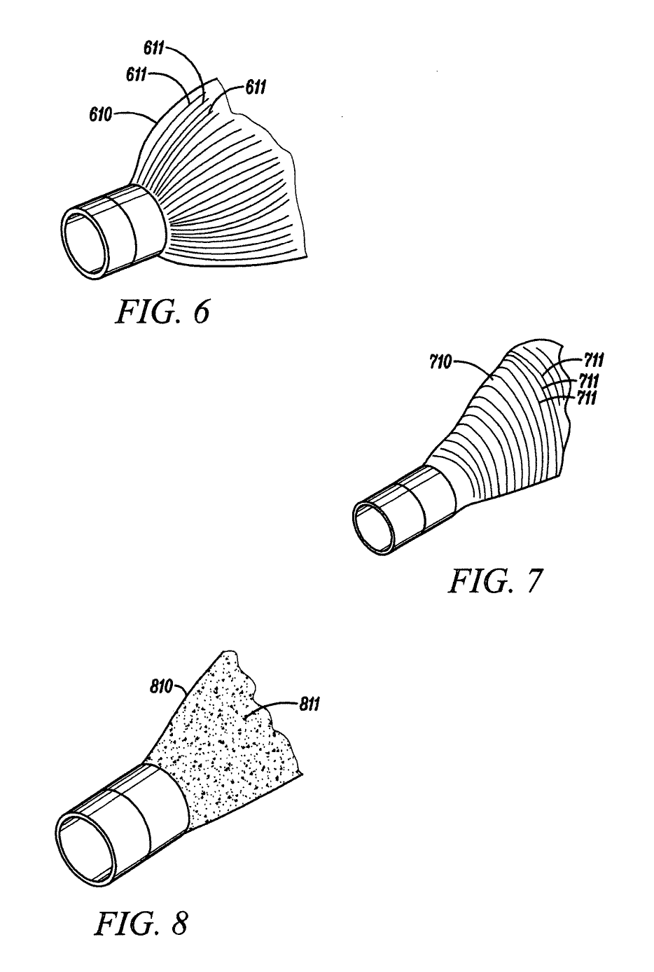

FIG. 6 shows a part of a fourth form of cuff;

FIG. 7 shows a part of a fifth form of cuff; and

FIG. 8 shows a part of a sixth form of cuff.

With reference first to FIGS. 1, the tracheostomy tube includes a tubular shaft 1 having a bore 2 extending along its length. The tube is formed with a relatively straight patient end portion 3 and a relatively straight machine end portion 4 linked by a curved intermediate portion 5 so that the patient and machine ends 6 and 7 are angled at about 100° to one another. The shaft 1 is extruded or moulded from a plastics material such as PVC. Towards its patient end 6 the tube has an inflatable sealing cuff 10.

At its machine end 7 the tube has a flange 20, to which a neck strap (not shown) is attached, and a standard 15 mm female tapered coupling 21.

The sealing cuff 10 is of the high-volume/low-pressure kind so that it has a relatively floppy shape when deflated but, when inflated, it fills out at low pressure to a diameter matching the internal diameter of the trachea T. The cuff 10 may be of PVC or any other conventional material. The cuff 10 is attached to the shaft 1 by two collar portions 12 and 13 at opposite ends. The cuff 10 extends over an opening 14 on the outer surface of the shaft 1 into an inflation lumen 15 extending along the shaft within its wall thickness. The inflation lumen 15 is connected towards the machine or rear end 7 of the tube with a small-bore inflation line 16, which is terminated by a combined inflation indicator or pilot balloon, valve and connector 17. The cuff 10 is inflated and deflated with air by connecting an air-filled syringe (not shown) with the connector 17. Alternatively, the cuff 11 could be inflated with a liquid, such as saline. Instead of being of a high-volume/low-pressure kind the cuff could be of a high-pressure elastic kind that is expanded and stretched by gas pressure supplied to the cuff.

The outer surface of the wall of the cuff 10 is formed with surface formations arranged to reduce the flow of secretions between the trachea and the cuff. In particular, the surface formations are chosen to interrupt any linear folds in the cuff that could provide a passage for secretions, or to make any path for secretions more tortuous. There are various forms of surface formations that could serve this function.

With reference first to FIG. 2 there is shown a part of a cuff 210 that is moulded with a pattern of small dimples or pimples 211 projecting outwardly and distributed over the entire surface of the inflatable portion of the cuff although it would be possible, instead, just to form these formations on that part of the surface that would come into contact with the wall of the trachea. The dimples 211 are located very close to one another, typically with a spacing equal to or less than the diameter of the dimples, which is between about 1 mm and 0.1 mm. The dimples 211 interact with one another to create a tortuous path that secretions would have to pass to reach the lower trachea, beneath the cuff. The dimple texture on the cuff 210 also allows the cuff to lie closer to the wall of the trachea because the raised dimples are able to fit into any dips and undulations in the wall of the trachea.

FIGS. 3 and 4 show a second, alternative cuff texture formed by a pattern of hatching 311 on the surface of the cuff 310. The hatching 311 is formed by two intersecting series of shallow, raised linear ridges 312 and 313 of triangular profile. Typically, these ridges 312 and 313 are about 0.1 mm wide and 0.1 mm high. The spacing between the parallel ridges in each set 312 and 313 is about the same as their width. The two series of ridges 312 and 313 could intersect at any angle, preferably between 45° and 135°, and most preferably around 90°. Instead of the hatching pattern being provided by ridges projecting outwardly of the cuff the pattern could be inverted to provide a similar pattern of indented channels 511 projecting inwardly, as illustrated by the cuff 510 shown in FIG. 5.

Instead of providing two sets of intersecting grooves or ridges, as shown in FIGS. 3 to 5, it would be possible just to provide a pattern of parallel, or substantially parallel, ridges or grooves.

FIG. 6 shows a cuff 610 with a pattern of linear grooves or ridges 611 extending longitudinally of the cuff. These grooves or ridges 611 would ensure that any folds that formed during inflation of the cuff would align with the grooves or ridges and cause the folds to be narrower than would otherwise be the case. This would mean that secretions would not be able to flow along the folds very easily, thereby reducing the amount of leakage past the cuff.

Alternatively, as shown in FIG. 7, the grooves or ridges 711 could extend circumferentially around the cuff 710. These grooves or ridges 711 would then act like a series of micro walls or ditches, providing an obstacle that the secretions would have to traverse in order to flow past cuff 710.

In a further, sixth embodiment, shown in FIG. 8, the outer surface of the cuff 810 is formed with a random, rough, uneven textured surface 811 similar to that produced by bead or shot blasting. The surface features preferably have dimensions of around 0.1 mm but could be less than this, such as between about 0.05 and 0.1 mm. The surface could either be provided by projections or indentations or by a combination of both projections and indentations. This surface allows the cuff 810 to conform better to the surface of the trachea, fitting into any imperfections in the trachea surface and around the hair-like cilia on the tracheal wall.

The invention is not confined to tracheostomy tubes but could be used on other tracheal tubes.

1-9. (canceled)

10. A tracheal tube having a shaft and an inflatable sealing cuff mounted on the shaft towards its patient end, characterised in that the cuff has a wall with an outer surface provided with surface formations adapted to reduce the flow of secretions past the cuff, and that the surface formations are provided by a pattern of intersecting linear projections or grooves on the wall surface.

11. A tracheal tube according to claim 10, characterised in that the dimensions of the surface formations are between 0.1 mm and 1 mm.

12. A tracheal tube according to claim 10, characterised in that the cuff is of the high-volume/low-pressure kind.

13. A cuff for a tracheal tube having a shaft, the cuff being an inflatable sealing cuff mounted on the shaft towards its patient end, the cuff having a wall with an outer surface provided with surface formations adapted to reduce the flow of secretions past the cuff, and that the surface formations are provided by a pattern of intersecting linear projections or grooves on the wall surface.

14. A cuff according to claim 13, wherein the dimensions of the surface formations are between 0.1 mm and 1 mm.

15. A cuff according to claim 13, wherein the cuff is of the high-volume/low-pressure kind.
